(19) United States
(12) Patent Application Publication
(10) Pub. No.: US 2019/0314076 A1
(43) Pub. Date: Oct. 17, 2019

(54) MODIFIED ENDOMETRIAL ABLATION

(71) Applicant: Arthur M. McCausland, Sacramento, CA (US)

(72) Inventor: Arthur M. McCausland, Sacramento, CA (US)

(21) Appl. No.: 16/380,817

(22) Filed: Apr. 10, 2019

Related U.S. Application Data
(60) Provisional application No. 62/655,962, filed on Apr. 11, 2018.

(51) Int. Cl. A61B 18/00; A61B 18/02; A61B 18/04; A61B 18/12

(52) U.S. Cl. CPC A61B 18/00; A61B 18/02; A61B 18/04; A61B 2018/1861; A61B 2018/00559; A61B 2018/00577; A61B 2018/0212; A61B 18/1206

(57) ABSTRACT

An endometrial ablation tool is configured for modified endometrial ablation, rather than global endometrial ablation. The tool can work according to various different methodologies including ablation by thermal energy (hot or cold), ablation by RF energy, ablation by microwave energy, ablation by plasma gas heating or ablation by other endometrial contact methodologies for ablation thereof. The ablation tool is shaped so that it avoids ablation in cornual areas. In one embodiment, the tool is configured with a unique posterior and anterior portion, with only one such portion configured to perform ablation, so that ablation occurs only with the anterior endometrium or only the posterior endometrium.

### CROSS-REFERENCE TO RELATED APPLICATIONS

This application claims benefit under Title 35, United States Code § 119(e) of U.S. Provisional Application No. 62/655,962 filed on Apr. 11, 2018.

### FIELD OF THE INVENTION

The following invention relates to endometrial ablation tools and methods, such as for treatment of menorrhagia. More particularly, this invention relates to endometrial ablation tools and methods which limit ablation to only portions of the endometrium, and especially avoiding ablation in cornual areas and lateral fundal wall areas.

### BACKGROUND OF THE INVENTION

Total resectoscopic and nonresectoscopic (global endometrial ablation—GEA) are minimally invasive surgical procedures developed as an alternative to hysterectomy for the treatment of intractable heavy menstrual bleeding (HMB), also known as menorrhagia. The goal of a total or global endometrial ablation is to destroy the endometrium in its entirety. This destruction exposes the intra-uterine myometrium. At the end of the procedure, after the ablation fluid and/or instruments are removed, these myometrial walls collapse and are in juxtaposition. Post ablation/resection myometrial inflammation and necrosis can potentially result in intra-uterine scarring and contracture. This intra-uterine scaring has been documented by multiple authors following both resectoscopic and nonresectoscopic ablations.

Turnbull et al. demonstrated on magnetic resonance imaging (MRI) that 95% of post ablation/resection patients have persistent or regenerating endometrium usually found in the upper fundal and/or cornual regions. If this tissue bleeds and is obstructed by post-ablation scarring/contracture, it can cause painful central hematometra, cornual hematometra, hematosalpinx (post ablation tubal sterilization syndrome=PATSS) or retrograde bleeding (potentially causing endometriosis).

Also, post-ablation scarring/contracture can make thorough evaluation of the intrauterine cavity difficult. The inability to make an accurate diagnosis when post ablation patients return with abnormal uterine bleeding (AUB), postmenopausal bleeding (PMB), or an abnormal imaging study is a major diagnostic dilemma. Post ablation scarring and contracture make endometrial biopsies, diagnostic hysteroscopy and saline infusion sonograms (SIS) unreliable. Ahonkallio et al. published a paper titled “Feasibility of Endometrial Assessment after Thermal Ablation.” They studied 57 patients 3-10 years after balloon or radiofrequency ablation. Transvaginal ultrasound was inconclusive because endometrial demarcation was not exact in any of the patients. Saline infusion sonograms were compromised because only 15% distended normally. Eighteen percent failed because the catheter could not be inserted. Twenty three percent of endometrial biopsies failed but tissue was successfully obtained in 77% of patients. The study referred to Turnbull's MRI paper and concluded that the biopsies do not always represent the entire endometrium because areas may not be accessible. The authors state that “The detection of malignancy in women who have undergone endometrial ablation may pose a problem, especially as typical early presentation of endometrial cancer with postmenopausal bleeding may not occur.” In their conclusion they say that endometrial ablation “significantly complicates endometrial assessment later on.” This is why endometrial thermal ablation cannot be recommended for patients with high risk factors for endometrial cancer.

### SUMMARY OF THE INVENTION

As with any new endometrial ablation technology, modifications are sometimes necessary in an attempt to minimize delayed complications caused by post GEA intrauterine scarring/contracture. A modified endometrial ablation (MEA) according to this invention, is defined as an ablation procedure designed to treat heavy menstrual bleeding without causing obstruction of the uterine cavity especially in the cornual region. This prevents any obstruction of bleeding that could potentially delay the diagnosis of endometrial cancer. It also avoids painful obstructed bleeding problems such as cornual hematometra, post ablation tubal sterilization syndrome (PATSS), or retrograde bleeding. In addition, it allows for future accurate endometrial cavity evaluation if a post MEA patient returns with AUB, PMB, pain or a suspicious imaging study.

MEAs may be performed by using one of two different techniques. In a first technique a heat, freezing or other GEA device is modified so it does not enter the cornual areas and limits injury to the upper lateral fundal wall at the top of the uterus. Using this technique, the cornual areas and upper lateral fundus are not injured. Scarring is prevented allowing these areas to remain open. In this technique, the endometrial cavity will be ablated causing it to contract into a tubular structure. Despite this contracture, the cavity remains open allowing access to the upper fundus and cornual areas which have not been ablated.

To accomplish this, in one embodiment a GEA balloon (delivering hot or cold thermal energy), flexible liner, stretchable silicone membrane, radiofrequency (RF), plasma heating, microwave heating or similar device is specifically modified not to enter into the cornual regions. It is also preferably modified to not ablate the upper lateral fundal wall at the top of the uterus. FIG. 1 illustrates an example of the current prior art balloon, flexible liner or similar global endometrial ablation (GEA) procedure. FIG. 2 illustrates an example of a prior art radio frequency (RF) or similar global endometrial ablation (GEA) procedure.

In a second technique, a heat, freezing or other GEA device is modified to ablate only the anterior or posterior endometrial wall and to also avoid the cornual areas. When only one wall is injured, it heals in juxtaposition to an uninjured endometrial surface on the opposite wall. Consequently, the injured and uninjured surfaces do not grow together. Unlike the above “first technique,” intrauterine scarring and contracture does not occur allowing for the cavity to be entirely open. This not only avoids any obstruction of the cornual areas but also allows for easy access if future endometrial cavity evaluation is necessary.

To accomplish this, a horizontal septum or other liner is placed in a GEA balloon or a flexible liner (or stretchable membrane, such as a silicone membrane). A heated or freezing solution is used on one side of the septum and a normal temperature solution on the opposite side. If a radiofrequency or similar GEA device is used, the liner is modified by insulating one side so that only one wall is ablated. All devices are modified not to enter the cornual areas similar to the “first technique” described above.

Since a MEA is designed not to destroy the entire endometrium, it will not reduce bleeding as much as a GEA. MEA's goal is to cause hypo or eumenorrhea, not amenorrhea. Although GEA manufacturers often define success with post-ablation amenorrhea rates, most women prefer not to have amenorrhea. Niles surveyed 400 women with HMB and found that 80% defined a “successful outcome” as hypo or eumenorrhea, not amenorrhea.

In conclusion, MEAs are designed to treat HMB without causing post GEA intrauterine scarring/contracture, especially in the cornual regions. This prevents any obstruction of bleeding that could potentially delay the diagnosis of endometrial cancer. It avoids painful obstructed bleeding problems such as cornual hematometra, post ablation tubal sterilization syndrome (PATSS), or retrograde bleeding. It also allows for future accurate endometrial cavity evaluation if a post MEA patient returns with AUB, PMB, pain or a suspicious imaging study.

### OBJECTS OF THE INVENTION

Accordingly, a primary object of the present invention is to provide an ablation tool sized and shaped to be insertable into a uterus for ablation of the endometrium, with the tool configured to avoid global endometrial ablation, but rather only ablating part of the endometrium.

Another object of the present invention is to provide an ablation tool configured to ablate portions of the endometrium of a uterus, but which avoids ablation of cornual areas.

Another object of the present invention is to provide an endometrial ablation tool which ablates only an anterior portion of the endometrium or a posterior portion of the endometrium.

Another object of the present invention is to provide an endometrial ablation tool which utilizes RF energy for endometrial ablation and which is configured to only partially ablate the endometrium, and especially avoid ablation within cornual areas.

Another object of the present invention is to provide an endometrial ablation tool which utilizes thermal energy for endometrial ablation and which is configured to only partially ablate the endometrium, and especially avoid ablation within cornual areas.

Another object of the present invention is to provide an RF energy endometrial ablation tool which is limited to only ablation of an anterior or a posterior portion of the endometrium.

Another object of the present invention is to provide a thermal energy endometrial ablation tool which is limited to only ablation of an anterior or a posterior portion of the endometrium.

Another object of the present invention is to provide an endometrial ablation method which can be used with a variety of different modified ablation tools, including RF energy ablation tools, thermal energy (hot or cold) ablation tools, and other contact based ablation methodologies to provide only partial endometrial ablation.

Another object of the present invention is to treat menorrhagia without blocking cornual areas.

Other further objects of the present invention will become apparent from a careful reading of the included drawing figures, the claims and detailed description of the invention.

### BRIEF DESCRIPTION OF THE DRAWINGS

FIG. 1 is a perspective sectional view of a human uterus with a prior art thermal energy ablation tool expanded therein, and depicting how prior art ablation tools extend into cornual areas of the uterus.

FIG. 2 is a perspective sectional view of a human uterus with a prior art RF energy endometrial ablation tool expanded therein, and depicting how prior art RF energy ablation tools extend into the cornual areas of the uterus.

FIG. 3 is a perspective sectional view of a human uterus with a modified endometrial ablation tool of this invention expanded therein and configured for use of thermal energy to partially ablate the endometrium, and specifically to avoid ablation in cornual areas.

FIG. 4 is a front elevation view of the thermal energy modified endometrial ablation tool of FIG. 3, and showing associated portions of the system, and with portions of the uterus shown in broken lines.

FIG. 5 is a perspective sectional view of a human uterus with a modified endometrial ablation tool of an embodiment of this invention expanded therein and configured for use of RF energy to partially ablate the endometrium, and specifically to avoid ablation in cornual areas.

FIG. 6 is a front elevation view of the RF energy modified endometrial ablation tool of FIG. 5, and showing associated portions of the system, and with portions of the uterus shown in broken lines.

FIG. 7 is a side elevation full sectional view of a human uterus with a semi-ablation thermal energy tool located therein and configured for posterior endometrial ablation.

FIG. 8 is a side elevation full sectional view of a human uterus with a semi-ablation thermal energy tool located therein and configured for anterior endometrial ablation.

FIG. 9 is a side elevation full sectional view of a human uterus with a semi-ablation RF energy tool located therein and configured for posterior endometrial ablation.

FIG. 10 is a side elevation full sectional view of a human uterus with a semi-ablation RF energy tool located therein and configured for anterior endometrial ablation.

### DESCRIPTION OF THE PREFERRED EMBODIMENT

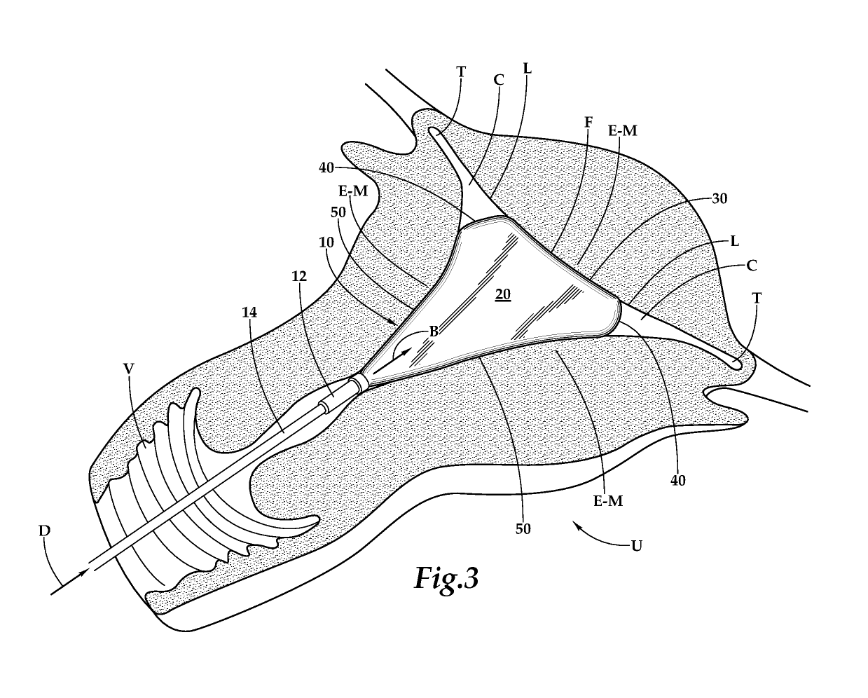

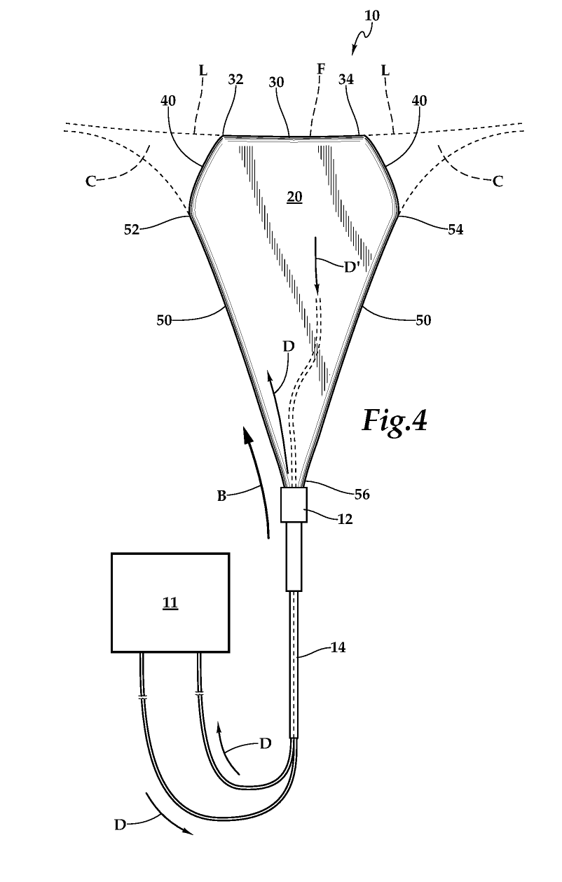

Referring to the drawings, wherein like reference numerals represent like parts throughout the various drawing figures, reference 10 (FIGS. 3 and 4) is directed to a thermal (or other) energy tool for use in performing modified endometrial ablation of an endometrium and superficial myometrium E-M of a uterus U. The tool 10 is configured to avoid ablation of cornual areas C of the uterus U. Furthermore, in certain embodiments the ablation tool can be configured to only provide ablation energy transfer to a posterior side, anterior side or other reduced portion of the endometrium and superficial myometrium E-M of the uterus U.

In essence, and with particular reference to FIGS. 3 and 4, basic details of the thermal energy tool 10 are described, according to a first embodiment. In this first embodiment, the tool 10 has a balloon 20 at a distal end of the tool 10, inserted into the uterus U. The balloon 20 includes an upper surface 30 configured to abut against a fundal wall F of the uterus U. This upper surface 30 extends laterally to truncated surfaces 40 which truncate the balloon 20 and keep the balloon 20 from extending into cornual areas C of the uterus U. Lower lateral surfaces 50 extend proximally from the truncated surfaces 40 back to an entry port 12 coupled to a delivery tube 14 from which the balloon 20 is extended. Fluid flow is caused to occur (along arrow D of FIGS. 3 and 4) to supply high (or low) temperature fluid into the balloon 20, with a temperature sufficient to cause sufficient heat transfer to result in ablation of endometrial tissues E-M (including the endometrium and superficial myometrium) where it is in contact with the surfaces 30, 40, 50 of the balloon 20. Cornual areas C and preferably also lateral fundal wall areas L are kept from experiencing ablation.

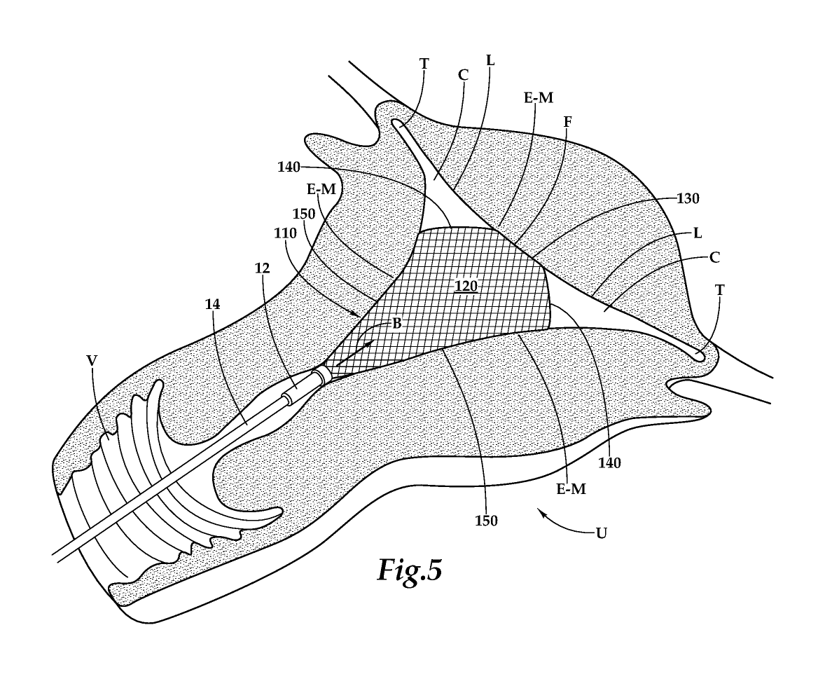

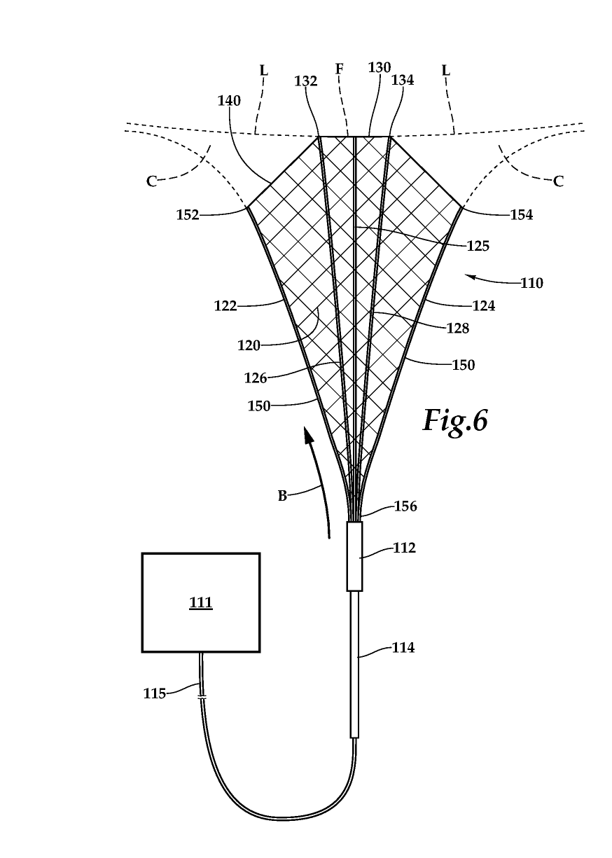

With reference to FIGS. 5 and 6, basic details of an RF energy tool 110 providing an alternative embodiment to the thermal energy tool 10, are described according to this alternative embodiment. The RF ablation tool 110 has a similar shape to the thermal energy tool 10, when the balloon 20 is expanded. The RF energy tool 110 is formed of a web mesh 120 of material configured to conduct the RF energy 110 and presented adjacent to the endometrial tissues E-M of the uterus U. This web mesh 120 of the RF energy tool 110 includes an upper surface 130 configured to abut the fundal wall F of the uterus U. This upper surface 130 extends laterally to truncated surfaces 140, which truncate the web mesh 120 and keep the web mesh 120 from extending into cornual areas C of the uterus U. Lower lateral surfaces 150 extend proximally from the truncated surfaces 140 back to an entry port 112 on a delivery tube 114, from which the RF energy tool 110 is extended. Electric energy is supplied to the web mesh 120 along the delivery tube 114 to energize the web mesh 120, and result in ablation of portions of the endometrial tissues E-M adjacent to the surfaces 130, 140, 150, while cornual areas C (and preferably also lateral fundal wall areas L) are kept from experiencing ablation.

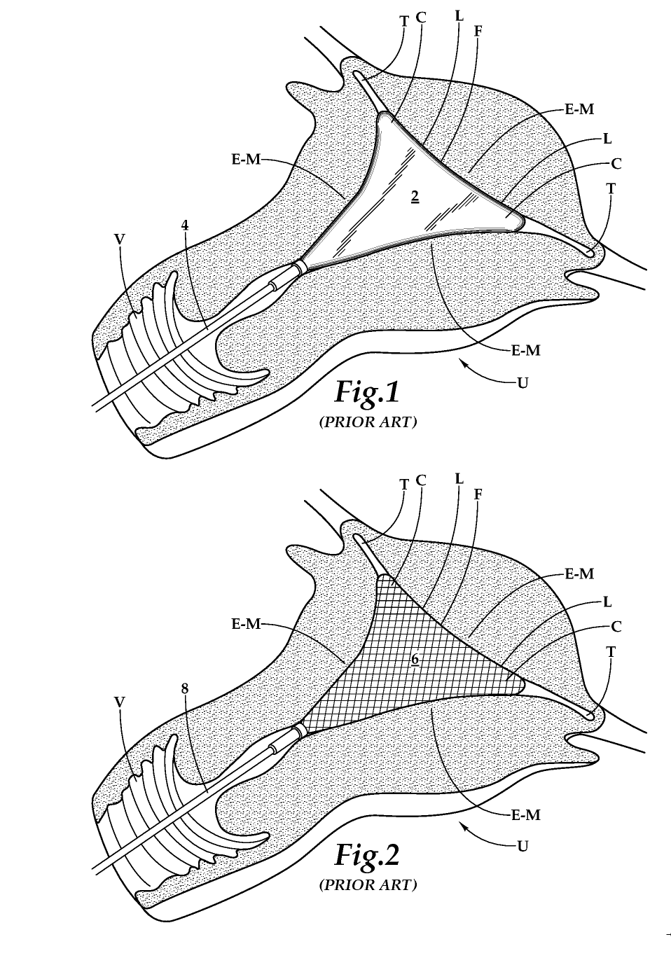

More specifically, and with particular reference to FIGS. 3 and 4, details of the thermal energy tool 10 are provided according to this first embodiment. Initially, applicant notes that in many respects this thermal energy tool 10 shares similarities with a prior art balloon 2 (FIG. 1) for use in thermal energy based endometrial ablation. Prior art balloons 2 are provided into the uterus U by first being supplied from a prior art delivery tube 4. The prior art balloon 2 includes lateral protuberances which extend deep into the cornual areas C, which lead to the fallopian tubes T. The prior art balloon 2 thus contacts as much of the endometrial tissues E-M as possible for total (or approaching total (also called global)) ablation of the endometrial tissues E-M.

As discussed in detail above, various complications can result from such total ablation of the endometrial tissues E-M, and most if not all therapeutic benefits can be provided by limiting ablation of the endometrial tissues E-M to being partial, and particularly to keeping the cornual areas C (and potentially also the lateral fundal wall areas L) free from ablation thereof. In other prior art embodiments, a prior art wire mesh 6 is delivered from a prior art delivery tube 8 and has a shape similar to that of the prior art balloon 2, but is configured to utilize RF energy for ablation of the entire endometrial tissues E-M (or near total (or global) ablation). Such prior art RF energy ablation tools with an associated wire mesh 6 are similarly configured with protuberances particularly to extend into cornual areas C for ablation of the endometrial tissues E-M adjacent thereto.

With this invention, the balloon 20 (generally including a flexible liner and/or a stretchable silicone (or other material) membrane) is particularly configured to leave the cornual areas C (and preferably also the upper lateral fundal wall areas L) free from ablation. Most preferably, the balloon 20 is configured with a shape that keeps the balloon 20 from extending into the cornual areas C. As an alternative, the balloon 20 can be sized smaller to achieve avoidance of contact with an entry into the cornual areas C. As a further alternative, the balloon 20 could extend into the cornual areas C, but a septum or other barrier could be provided within the balloon 20 to keep hot or cold ablation fluids from passing into such protuberances extending into the cornual areas C, so that ablation is limited to active portions of the balloon 20 which are truncated operationally to not extend ablation energy into the cornual areas C.

In the embodiment depicted herein (FIGS. 3 and 4) the balloon 20 includes truncated surfaces 40 which act as a preferred embodiment to keep the balloon 20 from extending into the cornual areas C. As an alternative, this truncated surface 40 could be an interior surface within the balloon 20 which merely keeps hot/cold fluids from extending past the truncated surface 40 and into the cornual areas C.

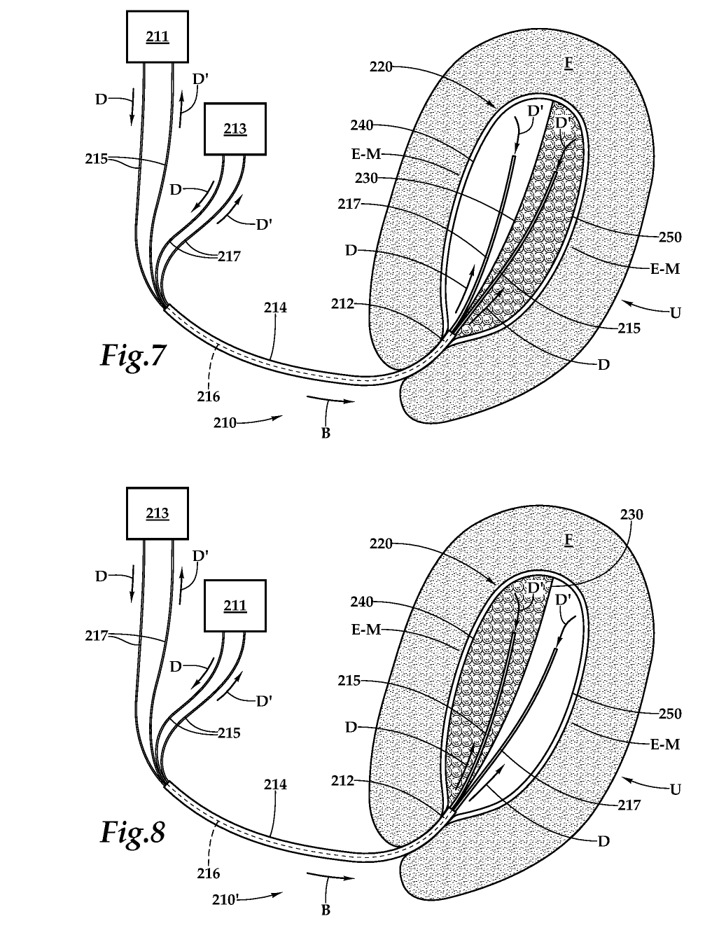

The balloon 20 preferably has a shape which is generally triangular with truncated corners when viewed posteriorly or anteriorly. When viewed laterally (i.e. for instance FIGS. 7 and 8), the balloon 20 has more of an oval shape. The balloon 20 is delivered from and supported by an entry port 12 and delivery tube 14 at a proximal end of the balloon 20. The delivery tube 14 extends proximally from an entry port 12 to an extreme temperature fluid source 11. This extreme temperature fluid source 11 can be a source of sufficiently hot or cold temperature fluid to result in heat transfer from surfaces of the balloon 20 at a sufficiently higher rate to cause ablation of the endometrial tissues E-M, where it is in contact with or nearby surfaces 30, 40, 50 of the balloon 20. The delivery tube 14 includes conduits therein, such as on either side of a divider wall. One side of the divider wall within the delivery tube 14 can supply extreme temperature fluid (along arrow D), while the opposite side of the divider wall can act as a return for the fluid after it has passed into the balloon 20, with this return (along arrow D′) leading back to the extreme temperature fluid source 11 for reheating (or re-cooling) before cycling back through the delivery tube 14, back to the interior of the balloon 20.

The delivery tube 14 also acts in placement of the balloon 20 within the uterus U. In particular, the balloon 20 is typically initially entirely retracted into an interior of the delivery tube 14 and on a proximal side of the entry port 12. The delivery tube 14 is then passed through the vagina and up into the uterus U. Once the entry port 12 extends into the uterus U, the delivery tube 14 is manipulated so that the balloon 20 is advanced distally from the delivery tube 14 (along arrow B) and into the uterus U. Such advancement typically continues until the upper surface 30 of the balloon 20 is adjacent to the fundal wall F of the uterus U. The balloon 20 is then inflated, such as with a fluid (typically liquid) until the balloon 20 has fully expanded, such as to the configuration depicted in FIGS. 3 and 4.

Once fully inflated, the upper surface 30 of the balloon 20 is adjacent to the fundal wall E Truncated surfaces 30 will have expanded laterally during expansion of the balloon 20, but stop short of passing into the cornual areas C, and preferably also stop short of coming into contact with or expanding beyond the upper lateral fundal wall L. Junctions between the upper surface 30 and the two truncated surfaces 40 are defined by a left lateral corner 32 and right lateral corner 34. Most preferably, these corners 32, 34 are spaced apart by a distance which is less than the spacing between the cornual areas C of the uterus U. Most preferably, the spacing between the corners 32, 34 is also sufficiently short that at least portions of the upper lateral fundal wall L are also left spaced from the truncated surfaces 40 of the balloon 20.

Lower lateral surfaces 50 of the balloon 20 extend from the truncated surfaces 40 back to the entry port 12. The lower lateral surfaces 50 taper toward each other as they extend from a left upper corner 52 adjacent to a left one of the truncated surfaces 40 and from a right upper corner 54 adjacent to a right one of the truncated surfaces 40. A lower edge 56 of the lower lateral surfaces 50 is generally cylindrically shaped and adjacent to the entry port 12. The lower lateral surfaces 50 have a tapering final shape so that when the balloon 20 is expanded, the lower lateral surfaces 50 generally maintain contact with the endometrial tissues E-M at middle portions of the uterus U. Preferably, the lower lateral surfaces 50 stop short of coming into contact with the cervix at the proximal end of the uterus U, leaving the cervix substantially unaffected by the ablation procedure.

After full placement and expansion of the balloon 20, extreme temperature fluid flow from the extreme temperature fluid source 11 is caused to occur (along arrows D and D′) into and out of the balloon 20. An extreme temperature of the fluid is sufficient to cause heat transfer either out of the fluid and into the endometrial tissues E-M or out of the endometrial tissues E-M and into the fluid (in the case of an extreme low temperature fluid). Heat transfer is at a sufficiently higher rate that it causes ablation of the endometrial tissues E-M. Such ablation is to some extent a function of time and to some extent a function of the particular temperature of the extreme temperature fluid. Through appropriate experimentation or other design techniques, an optimal combination of temperature and time can be selected to optimize the ablation procedure.

After the ablation procedure is completed, the fluid is removed from the balloon 20 (at least partially), along the delivery tube 14. The balloon 20 can then be retracted into the delivery tube 14 through the entry port 12 (in a direction opposite that of arrow B) and the delivery tube 14 (along with the balloon 20 contained therein), can be translated proximally and removed from the uterus U. As an alternative, the balloon 20 can remain deployed from the entry port 12, but at least partially deflated by at least partial removal of fluid from an interior of the balloon 20, and retracted proximally to remove the delivery tube 14 and balloon 20 simultaneously from the uterus U without (or with only partial) retraction.

With particular reference to FIGS. 5 and 6, specific details of the RF energy tool 110 are described, according to one form of this alternative embodiment. The RF energy tool 110 includes an entry port 112 and delivery port 114 which function similar to the entry port 12 and delivery tube 14 for the thermal energy tool 10. An RF energy source 111 has a wire 115 extending therefrom which passes through delivery tube 114 and is coupled to the web mesh 120 of the RF energy tool 110. This web mesh 120 is preferably initially retracted within delivery tube 114, so that, once the entry port 112 has been passed into the uterus U, the web mesh 120 can be advanced distally out of the delivery tube 114 through the entry port 112, to expand into the uterus U (along arrow B of FIGS. 5 and 6).

The web mesh 120 is formed of sufficiently electrically conductive materials that the wire 115 from the alternative energy source 111 causes heating of the web mesh 120 when appropriate electric power and RF energy is supplied through the wire 115 from the RF energy source 111 to the web mesh 120. Such heating of the web mesh 120 is sufficiently great that portions of the endometrial tissues E-M of the uterus U adjacent to the web mesh 120 are caused to undergo ablation.

The web mesh is configured (either through a particular shape or a particular size or both) to avoid ablation of cornual areas C, and preferably also to avoid ablation of the endometrial tissues E-M adjacent to the upper lateral fundal wall L adjacent to each of the cornual areas C. In this particular embodiment, the web mesh 120 is shaped to avoid delivery of RF energy at a sufficiently high level to cause ablation within the cornual areas C. In particular, the web mesh 120 includes an upper surface 130 configured to be placed adjacent to the fundal wall F of the uterus U. A pair of truncated surfaces 140 extend diagonally both laterally and proximally from the upper surface 130. A lower lateral surface 150 extends from the truncated surfaces 140 back proximally to a lower edge 156 adjacent to the entry port 112 of the delivery tube 114.

In this particular embodiment, the web mesh 120 is provided with the desired shape by utilizing a central rib 125 along with a left lateral rib 122 and a right lateral rib 124, and with the left mid rib 126 between the left lateral rib 122 and the central rib 125. A right mid rib 128 is provided between the right lateral rib 124 in the central rib 125. The web mesh 120 both includes these ribs 122, 124, 125, 126, 128 and also preferably also includes smaller wires spanning between these ribs 122, 124, 125, 126, 128. These ribs can be formed of electrically conductive material or can merely be provided as structural elements, with only the mesh wiring supported by the ribs being electrically conductive.

Most preferably each of the ribs 122, 124, 125, 126, 128 is formed of a material which has a shape memory but is also flexible to an extent short of an elastic limit of the material. Thus, these ribs can flex to cause the ribs to come together within the delivery tube 114. However, when the web mesh 120 is advanced out of the delivery tube 114 (along arrow B of FIGS. 5 and 6) the ribs are free to transition to a previous shape memory shape which has been imparted to each of the ribs in advance. For instance, the left lateral rib 122 and right lateral rib 124 can be pre-formed to have a shape memory for a shape which places these lateral ribs 122, 124 adjacent to left and right lateral sides of the uterus U. Correspondingly, the mid ribs 126, 128 can be provided with a shape memory which causes these mid ribs 126, 128, when released from the delivery tube 114, to attain a position which is only slightly bent and midway between the lateral ribs 122, 124 and the central rib 125.

While each of these ribs is shown as a singular rib, if desired, each of the singular ribs depicted in FIG. 6 could be provided in a pair including an anterior rib and a posterior rib, to keep portions of the wire mesh 120 adjacent to both anterior and posterior sides of the uterus U (see for instance FIGS. 7-10). In such an embodiment, the wire mesh 120 would include anterior and posterior sides of the wire mesh 120 which are together adjacent to all of the endometrial tissues E-M, other than the cornual areas C (and preferably also the upper lateral fundal wall L).

The upper surface 130 of the wire mesh 120 preferably extends laterally until it terminates at a left lateral corner 132 and a right lateral corner 134. These lateral corners 132, 134 are preferably spaced apart by sufficiently short distance that the upper lateral fundal wall L is left free from contact with the web mesh 120. Most preferably, the mid ribs 126, 128 have a shape memory which causes them to extend to these lateral corners 132, 134.

Proximal portions of the truncated surfaces 140 transition into the lower lateral surfaces 150 through a left upper corner 152 and right upper corner 154. Remaining portions of the lower lateral surfaces 150 extend somewhat as a funnel shape down to the lower edge 156 where the lower lateral surfaces 150 can together be generally cylindrical in form directly adjacent to the entry port 112.

With the shape configuration described above, the RF energy tool 110 can be activated and have RF energy 111 delivered along the wire 115 to the web mesh 120, causing portions of the endometrial tissues E-M adjacent to the web mesh 120 to be ablated. Importantly, the cornual areas C, and preferably also portions of the upper lateral fundal wall L, are not ablated because the web mesh 120 is not adjacent thereto. Ablation is achieved by a combination of an appropriate amount of RF energy delivered by the RF energy tool 110, as well as an amount of time that the RF energy tool 110 is in operation. The RF energy level and time can be increased or decreased to optimize extent of ablation which occurs to the endometrial tissues E-M, and to otherwise achieve optimal results. Thereafter, the RF energy tool 110 can be removed from the uterus U by translation proximally, either with or without first retracting the wire mesh 120 back into the delivery tube 114 (in a direction opposite arrow B).

With particular reference to FIGS. 7 and 8 a semi-ablation thermal energy tool 210 is described, according to an alternative embodiment. Many of the details of this semi-ablation thermal energy tool 210 are similar to those of the thermal energy tool 10 described in detail above. Uniquely, the semi-ablation thermal energy tool 210 is configured for only ablation of either a posterior side of the uterus U (FIG. 7) or an anterior side of the uterus U (FIG. 8). The tool 210 includes an extreme temperature fluid source 211 and a normal temperature fluid source 213 which are coupled through first conduits 215 and second conduits 217 passing through the delivery tube 214 and through the entry port 212 to anterior and posterior sides of an interior of the balloon 220.

A pair of divider walls 216 are provided within the delivery tube 214 to split the delivery tube 214 into a pair of first conduits 215 and a pair of second conduits 217. Alternatively, four separate conduits 215, 217 can be nested within the delivery tube 214 (or some sub-combination thereof can be provided). The goal of the conduits 215, 217 and the divider walls 216 is for delivery of an extreme temperature fluid and for return of the extreme temperature fluid relative to the extreme temperature fluid source 211, and to separately allow for flow of a normal temperature fluid from the normal temperature fluid source 213 into and out of a separate half of the balloon 220.

A septum 230 or other barrier wall is provided within the balloon 220 which divides an interior of the balloon 220 into an anterior half and a posterior half. The balloon 220 has an anterior surface 240 opposite a posterior surface 250. One of these surfaces is active for ablation, and an operator can select, in a preferred embodiment, which of these surfaces 240, 250 is the active ablation surface. The active ablation surface would have the extreme temperature fluid from the extreme temperature fluid source 211 passing thereinto and returning therefrom (along arrows D, D′). On the opposite side of the septum 230, normal temperature fluid is circulated, also shown on arrows D, D′. Thus, the normal temperature fluid causes one of the surfaces 240, 250 to have no ablation occur while the other surfaces 240, 250 are in contact with the extreme temperature fluid from the extreme temperature fluid source 211 and causes ablation of the endometrial tissues E-M on either the anterior or posterior side of the uterus U. In FIG. 7, the posterior side of the uterus U is ablated by heat transfer through the posterior surface 250. In FIG. 8, the anterior side of the uterus U is caused to experience ablation through the anterior surface 240 heat transfer. Other details of the semi-ablation thermal energy tool 210 are similar to those of the thermal energy tool 10 described in detail above.

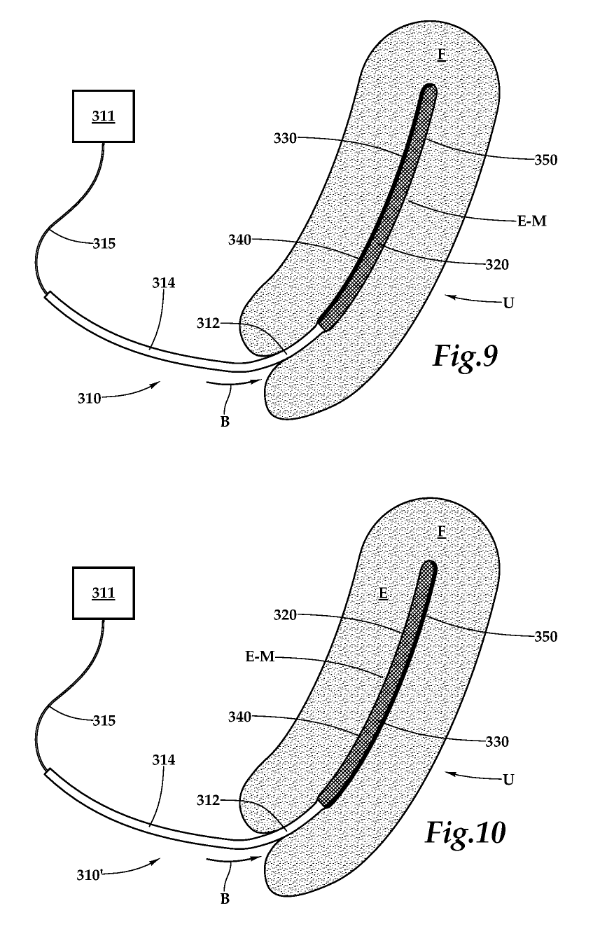

With particular reference to FIGS. 9 and 10, a semi-ablation RF energy tool 310 is described. This tool 310 is a modified version of the RF energy tool 110 described above, but configured for ablation of either an anterior endometrial wall or a posterior endometrial wall, while the opposite wall remains free of ablation. The tool 310 includes an entry port 312 at a distal end of a delivery tube 314. An RF energy source 311 is coupled to a wire 315 (or bundle of wires) which lead to a web mesh 320 deployable distally from the entry port 312 of the delivery tube 314.

The web mesh 320 preferably has a shape similar to that of the web mesh 120 of the RF energy tool 110 described in detail above. However, an insulating liner 330 is provided upon an anterior surface 340 of the web mesh 320 or upon the posterior surface 350 of the web mesh 320. This insulating liner 330 has sufficient insulating characteristics to keep energy such as RF energy and/or heat from passing therethrough with sufficient magnitude to cause ablation. Rather, the insulating liner 330 abuts a posterior or anterior side of the uterus U which does not undergo any ablation. The anterior surface 340 or posterior surface 350 opposite the insulating liner 330 is provided with the web mesh 320 coupled to the RF energy source 311, to cause ablation of an adjacent anterior or posterior wall of the uterus U.

In one embodiment, the same semi-ablation RF energy tool 310 can be provided for either anterior surface ablation or posterior surface ablation, by reversing the tool 310 before insertion. As an alternative, the tool 310 can be optimized, such as with appropriate curvature, so that a separate tool 310 would be provided for anterior surface 340 ablation by RF energy delivery from the wire mesh 320, or posterior surface 350 ablation by passage of RF energy to the wire mesh 320 adjacent to the posterior surface 350 of the tool 310. Other details of the semi-ablation RF energy tool 310, including the contour and configuration of the semi-ablation RF energy tool 310, can be similar to that described above with respect to the RF energy tool 110 (FIGS. 5 and 6).

Various embodiments have been provided to illustrate how modified endometrial ablation can occur through utilization of a particularly configured ablation tool, and particularly a tool which avoids cornual areas C of the uterus U, and optionally to preferably also avoiding upper lateral fundal wall L portions of the uterus U. In addition, and as an option, modified endometrial ablation can be provided by utilizing a semi-ablation tool 210, 310 which is only active at an anterior wall or posterior wall of the uterus U, for ablation of only a portion of the endometrial tissues E-M, either on the anterior wall or the posterior wall of the uterus U. With such a semi-ablation tool 210, 310, the configuration of the tools 210, 310 is still sufficiently limited so that it avoids ablation within the cornual areas C, and preferably also avoids ablation at the upper lateral fundal wall L adjacent to the cornual areas C.

The principles embodied in these above described embodiments can also be extended to other endometrial ablation technologies. For instance, in one such system argon gas or some other easily ionizable gas is provided within a balloon or other membrane and an electric current is applied to this gas, causing the gas to turn into a plasma and to heat the balloon or other membrane sufficiently to cause thermal ablation to occur. Such a plasma ablation system could be provided within a balloon which has a shape configured to avoid cornual areas C, according to this invention. In other embodiments where microwave energy is utilized, a tool delivering the microwave energy can be provided with appropriate spacers which prevent movement of the microwave energy wand from extending laterally into the cornual areas. Furthermore, a shape of the tool and/or usage techniques can be provided to concentrate microwave radiation energy nearest a center of the uterus U, so that ablation energy does not extend into the cornual areas.

This disclosure is provided to reveal a preferred embodiment of the invention and a best mode for practicing the invention. Having thus described the invention in this way, it should be apparent that various different modifications can be made to the preferred embodiment without departing from the scope and spirit of this invention disclosure. When embodiments are referred to as “exemplary” or “preferred” this term is meant to indicate one example of the invention, and does not exclude other possible embodiments. When structures are identified as a means to perform a function, the identification is intended to include all structures which can perform the function specified. When structures of this invention are identified as being coupled together, such language should be interpreted broadly to include the structures being coupled directly together or coupled together through intervening structures. Such coupling could be permanent or temporary and either in a rigid fashion or in a fashion which allows pivoting, sliding or other relative motion while still providing some form of attachment, unless specifically restricted.

What is claimed is:

1: A method for performing modified endometrial ablation, including the steps of:
placing a modified endometrial ablation tool into a uterus, the modified endometrial ablation tool having a configuration which keeps ablating portions thereof spaced away from cornual areas of the uterus;
activating the modified endometrial ablation tool to ablate at least portions of walls of the endometrium spaced away from the cornual areas; and
removing the modified endometrial ablation tool from the uterus.

2: The method of claim 1 wherein said placing step includes a shape of the modified endometrial ablation tool causing cornual areas to be avoided.

3: The method of claim 1 wherein said placing step includes a size of the modified endometrial ablation tool causing cornual areas to be avoided.

4: The method of claim 1 wherein said activating step includes the tool delivering radio frequency radiation therefrom.

5: The method of claim 1 wherein said activating step includes the tool delivering freezing temperature structures for ablation of endometrial tissues by freezing.

6: The method of claim 1 wherein said activating step includes the tool delivering microwave energy radiation therefrom.

7: The method of claim 1 wherein said activating step includes the tool delivering hot temperature fluid for ablation of endometrial tissues by burning.

8: A modified endometrial ablation tool, comprising in combination:
at least one ablation surface taken from the group of surfaces including an anterior surface and a posterior surface;
an ablation energy supply coupled to said ablation surface; and
said ablation surface having a configuration which avoids cornual areas when placed within a uterus.

9: The tool of claim 8 wherein said ablation surface is shaped to avoid cornual areas.

10: The tool of claim 8 wherein said ablation surface is sized to avoid cornual areas.

11: The tool of claim 8 wherein said ablation energy supply includes a source of radio frequency radiation energy.

12: The tool of claim 8 wherein said ablation energy supply includes a below 0° C. temperature heat sink, sufficiently cold to ablate endometrial tissues by freezing.

13: The tool of claim 8 wherein said ablation energy supply includes a source of microwave radiation energy.

14: The tool of claim 8 wherein said ablation energy supply includes a source of hot fluid sufficiently hot to ablate endometrial tissues by burning.

15: The tool of claim 8 wherein only one of said anterior surface and said posterior surface is configured as an ablation surface.

16: A method for performing a modified endometrial ablation procedure, the method including the steps of:
placing a modified endometrial ablation tool into a uterus, the modified endometrial ablation tool having a configuration which keeps ablating portions thereof spaced away from cornual areas of the uterus;
activating the modified endometrial ablation tool to ablate at least portions of walls of the endometrium spaced away from the cornual areas;
removing the modified endometrial ablation tool from the uterus;
wherein the tool includes an anterior surface and a posterior surface, only one of the surfaces being at least part of the ablating portions of the tool; and
wherein said activating step ablates the endometrium on only a posterior portion or an anterior portion of the uterus.

17: The method of claim 16 where said placing step includes the tool delivering radio frequency radiation therefrom.

18: The method of claim 16 wherein said activating step includes the tool delivering freezing temperature structures for ablation of endometrial tissues by freezing.

19: The method of claim 16 wherein said activating step includes the tool delivering microwave energy radiation therefrom.

20: The method of claim 16 wherein said activating step includes the tool delivering hot temperature fluid for ablation of endometrial tissues by burning.
